(19) United States
(12) Patent Application Publication
(10) Pub. No.: US 2022/0031876 A1
(43) Pub. Date: Feb. 3, 2022

(54) VEHICLE SEAT WITH DISINFECTING LIGHT

(71) Applicant: FAURECIA AUTOMOTIVE SEATING, LLC, Auburn Hills, MI (US)

(72) Inventor: Sachin UBALE, Troy, MI (US)

(21) Appl. No.: 16/983,066

(22) Filed: Aug. 3, 2020

(51) Int. Cl. A61L 2/10; A61L 2/24; B60N 2/58; B60N 2/90

(52) U.S. Cl. CPC A61L 2/10; A61L 2/24; A61L 2202/16; B60N 2/986; A61L 2202/14; B60N 2/58

(57) ABSTRACT

A vehicle seat includes a disinfecting light source configured to kill or inactivate bacteria, viruses, or other pathogens residing on a seating surface or on other surfaces adjacent the seat. A controller ensures the vehicle is not occupied while a disinfection cycle takes place. The light source can be in strip form and integrated with a decorative covering of the seat as part of its aesthetic design.

### TECHNICAL FIELD

The present disclosure relates to vehicle seats.

### BACKGROUND

Sanitizing or disinfecting surfaces of a vehicle interior has traditionally been limited to application of an external disinfectant, such as spraying with a disinfecting aerosol or wiping surfaces with a disposable cloth saturated with disinfectant. Such techniques are problematic with cloth seating surfaces and pose an inconvenience to vehicle users, particularly in an age of ride-sharing, where a user may be forced to decide between taking a health risk or spending the time and money to always have external disinfectants handy for the next time they enter a vehicle.

Chinese patent publication CN 108839596 discloses a vehicle seat with a sanitizable headrest. The headrest has a fabric covering that slides along and around an underlying headrest body and into a cavity in the top of the seat back. Germicidal lights concealed within the cavity shine on the portion of the fabric slid into the cavity. The next time the fabric covering is slid around the headrest body, the sanitized portion is brought out to the exterior of the seat for use by a passenger and the previously used portion is slid into the cavity with the germicidal lights. The invention does not address areas of the seat more likely to be touched by the hands, legs, or torso of a seat occupant.

### SUMMARY

In accordance with one or more embodiments, a vehicle seat includes an external disinfecting light source.

In one or more embodiments, the vehicle seat includes a seating surface, and light from the light source is directed at the seating surface.

In one or more embodiments, light from the light source is directed away from the seat toward a vehicle interior panel when installed in a vehicle.

In one or more embodiments, light from the light source is not directed above a belt line of a vehicle when installed in the vehicle.

In one or more embodiments, the light source is located along a bolster section of the seat.

In one or more embodiments, light from the light source is directed at a bolster section of the seat and toward a centerline of the seat.

In one or more embodiments, the light source is sewn to a decorative covering of the seat.

In one or more embodiments, the light source is a strip of disinfecting lights.

In one or more embodiments, the light source is a flexible tube light.

In one or more embodiments, a first portion of the light source is transversely spaced from a second portion of the light source, and the first and second portions emit light toward each other.

In one or more embodiments, the vehicle seat includes a seat bottom and a seat back, and the light source comprises a plurality of portions transversely spaced along the seat bottom and the seat back.

In one or more embodiments, light from the light source includes ultraviolet light.

In one or more embodiments, light from the light source includes visible light.

In one or more embodiments, the light source is prevented from emitting light when a vehicle in which the seat is installed is occupied.

In one or more embodiments, the light source is activated remotely.

Various aspects, embodiments, examples, features and alternatives set forth in the preceding paragraphs, in the claims, and/or in the following description and drawings may be taken independently or in any combination thereof. For example, features disclosed in connection with one embodiment are applicable to all embodiments in the absence of incompatibility of features.

### BRIEF DESCRIPTION OF THE DRAWINGS

Exemplary embodiments will hereinafter be described in conjunction with the appended drawings, wherein like designations denote like elements, and wherein:

FIG. 1 is a perspective view of a vehicle seat with an integrated disinfecting light source;

FIG. 2 is a cross-sectional view of a seat bottom of the vehicle seat;

FIG. 3 is an enlarged view of a portion of FIG. 2;

FIG. 4 is a cross-sectional view of the seat bottom with the light source alternatively located;

FIG. 5 is an enlarged view of a portion of FIG. 4; and

FIG. 6 is a side view of the vehicle seat installed in a second row of seats with the light source alternatively located.

### DETAILED DESCRIPTION

Described below are embodiments of a vehicle seat equipped to self-disinfect and/or to disinfect vehicle interior panels arranged around the seat in the vehicle passenger cabin. As used herein, to disinfect means to destroy or otherwise inactivate bacteria, viruses, or other pathogens residing on a surface.

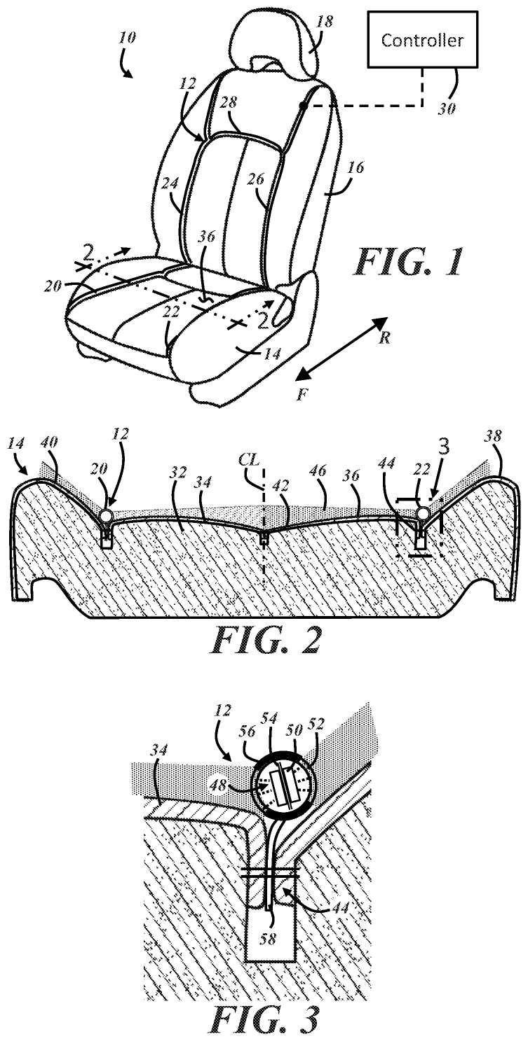

FIG. 1 illustrates an example of a vehicle seat 10 that includes a disinfecting light source 12. The light source 12 is an integral part of the seat 10 and is arranged along the exterior of the seat. The seat 10 includes a seat bottom 14 that supports a seat occupant from beneath and a seat back 16 that supports the upper body of the seat occupant from behind. The seat may include a headrest 18 at the end of the seat back 16. Forward and rearward directions (F, R) are indicated in FIG. 1 and are relative to the seat. The seat also has perpendicular transverse (left-to-right) and vertical directions. In the illustrated embodiment, the light source 12 includes transversely spaced portions 20, 22 along the seat bottom 14, transversely spaced portions 24, 26 along the seat back 16, and a transversely extending portion 28 along the seat back. The light source 12 is powered by the electrical system of the vehicle with wiring concealed within and/or beneath the seat 10. A controller 30 may be provided to selectively power and unpower the light source 12. The controller 30 may be part of the seat 10 or part of another vehicle system and is configured to receive information from sources internal or external to the vehicle and to make a determination whether or not the light source 12 should receive power at any given time.

FIG. 2 is a cross-sectional view of the seat bottom 14 of FIG. 1, which includes a cushion 32 and a decorative covering 34 (e.g., leather or cloth upholstery). The seat bottom 14 may include other components, such as structural supports, heating elements, air flow channels, etc. that are not shown here for the sake of simplicity in explanation. The decorative covering 34 provides a seating surface 36 with which a seat occupant is in contact while seated. The seat bottom 14 includes transversely spaced left and right bolster sections 38, 40 with a center section 42 spanning the space therebetween. The seating surface 36 includes the exterior surface of the covering 34 over the center section 42, further extending from opposite sides of the center section and over the tops of the respective bolster sections 38, 40.

The light source 12 is located along each of the bolster sections 38, 40. In this embodiment, one portion 20 of the light source 12 is located along a boundary between the right bolster section 40 and the center section 42, and another portion 22 of the light source is located along a boundary between the left bolster section 38 and the center section. In the illustrated example, the decorative covering 34 is of the cut-and-sew variety, and each portion 20, 22 of the light source 12 is located along a seam 44 of the covering. The light source 12 may be affixed along the exterior of the seat 10 by any suitable means. Preferably, at least a portion of the light source 12 is sewn to the decorative covering 34 to provide a robust attachment that can withstand multiple entries and exits of seat occupants over the life of the seat 10.

Light 46 from the light source 12 is directed at the seating surface 36. In this example, light from each of the transversely spaced portions 20, 22 of the light source 12 is directed inwardly toward a center line (CL) of the seat and outwardly over the bolster sections 38, 40. Disinfection of the seating surface 36 occurs wherever the light 46 impinges the seating surface. The light source 12 is configured to produce disinfecting light, which is able to kill or otherwise inactivate pathogens without the use of chemicals. The light 46, for example, includes ultraviolet light having a wavelength in a range from 100 nm to 400 nm. The ultraviolet light can be broad spectrum light having a wavelength in a range from 200 nm to 400 nm. In one embodiment, the light is UVC light having a wavelength in a range from 100 nm to 280 nm.

The light 46 may also include visible light having a wavelength in a range from 400 nm to 700 nm. Ultraviolet light is non-visible, and including visible light in the spectrum of light emitted by the light source 12 has certain advantages, such as emission of a visible glow when the light source is powered. This can provide a user with a visible indication that the disinfecting light source 12 has been activated, along with a similarly visible indication that the disinfecting cycle is over when the glow is extinguished. The inclusion of visible light also provides a desirable aesthetic from outside of the vehicle, making the vehicle appear to have neon-lighted seat trim, for example. Vehicle interior designers are thus provided an added visual tool for designing the look and feel of the vehicle, particularly in dark environments.

The light source 12 can be in strip or tube form with a plurality of individual LEDs configured to produce the disinfecting light 46 spaced along its length. A strip of LEDs may include LEDs along its length spaced every 10 mm to 40 mm, for example. One non-limiting example is illustrated in the enlarged view of FIG. 3, where a strip 48 of LEDs 50 is encased in a flexible plastic tube 52, which is at least partially transparent to the wavelengths of light produced by the LEDs. The LEDs 50 are arranged along a flexible polymer film 54 that serves as a flexible circuit board that electrically interconnects the LEDs along the length of the strip. In this example, there are LEDs on both opposite facing sides of the film 54.

An optional mask or shield 56 is included along the length of the light source 12 to block light along a portion of the circumference of the tube 52. The mask 56 may be an opaque portion that limits the amount of disinfecting light transmitted away from the seating surface 36. The mask 56 can be a film (e.g., paint or adhesively attached) along a wall of the tube 52, or the tube 52 may be extruded with an opaque stripe. Depending on the particular wavelengths of light being used for disinfection, it may be important to limit the amount of stray light for safety reasons—i.e., in some cases, persons should not be permitted to look directly at the light being emitted from the light source 12. In some embodiments, the seat is configured so that light 46 from the light source 12 is not directed a belt line of the vehicle. The beltline of the vehicle is defined at the lower edge of any window openings of the vehicle.

FIG. 3 also illustrates one non-limiting example of the light source 12 being sewn to the decorative covering 34. In this example, the light source 12 includes an attachment tab 58 which is sewn together with the pieces of decorative covering material as part of the seam 44. The tab 58 can be coextruded as one solid piece with the tube 52, or it may be a separately formed piece attached by other means such as adhesive, welds, or fasteners. The tab 58 can be continuous along the entire length of the tube 52, or it may be segmented with discrete tab pieces spaced out along the length of the light source 12. In one embodiment, the attachment tabs 58 are narrow fabric loops that wrap around the tube 52 to be sewn together with the seam 44.

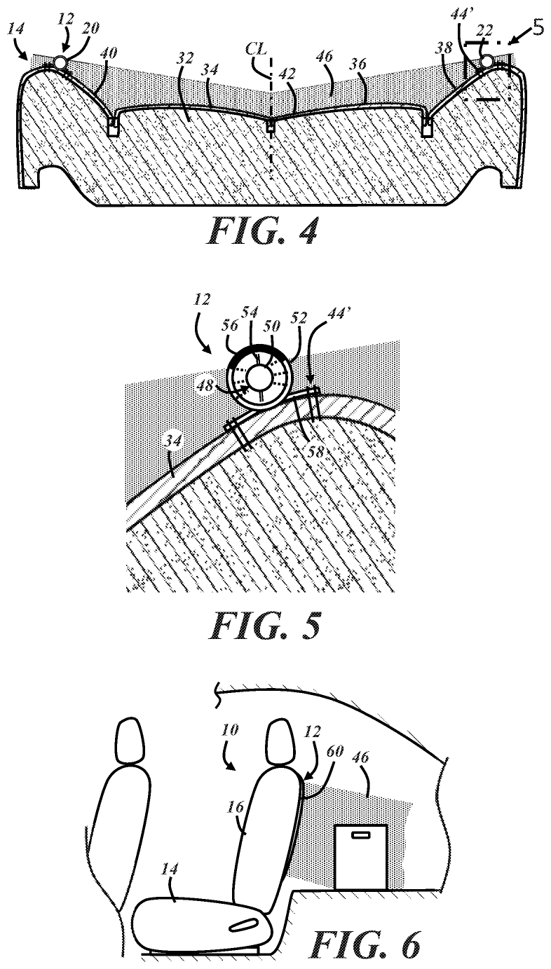

FIGS. 4 and 5 illustrate another example of the seat bottom 14 in which the portions 20, 22 of the light source 12 are attached to the decorative covering 34 by top-stitching 44′ along the bolster sections 38, 40 at locations spaced away from the center section 42 and vertically higher than in the example of FIGS. 1-3. In this example, most of the seating surface 36 being disinfected is between the transversely spaced portions 20, 22 of the light source, which is impinged by light 46 that is directed inwardly toward the center line (CL) of the seat. A smaller portion of the seating surface 36 is disinfected by light directed outwardly over part of the bolster sections 38, 40.

The light source 12 in this example also includes a strip 48 of LEDs 50 spaced along the length of a flexible polymer film 54 and encased in a flexible plastic tube 52. In this case, the LEDs 50 are round and pass through openings in the flexible plastic film 54 so that light from the same LED is emitted in all radial directions. The mask 56 in this case runs only along the top side of the tube 52. The attachment tab 58 in this example runs tangential to the tube 52, as opposed to the radial attachment tab of FIG. 3. Since the light source 12 is not located at an already existing seam of the decorative covering 34, it is sewn to the decorative covering via top-stitching 44′. Top-stitching can be purely functional, or it can add a decorative element, such as contrasting color or a decorative pattern. Innovative forms of top-stitching are disclosed in co-owned U.S. Pat. No. 8,312,826 (Lafferty et al.), and co-owned U.S. Pat. Nos. 8,100,070, and 8,316,785 (Boinais et al.). Other innovative decorative stitching techniques, some of which are also functional, are disclosed in U.S. Pat. Nos. 8,579,347 and 8,678,458 and 8,721,821 (Filipp et al.), and in U.S. Pat. No. 8,714,626 (Kornylo).

The illustrated light sources, attachment tabs, strips of LEDs, etc. are merely illustrative, as there are countless variations that fall within the scope of the appended claims. For example, the strips of lights need not be encased in a cylindrical tube. The tube can be in any shape and, in some cases, is not a tube at all. The LEDs or other light producing elements may for example be overmolded in a clear flexible material (e.g., silicone) having a cross-section of any shape. In some cases, the illustrated tubes are replaced with fiber optic or other types of light guides that allow the disinfecting light to travel from a remote source and along the light guide to be emitted at the exterior of the seat.

Skilled artisans with also appreciate that the cross-sectional views of the seat bottoms in FIGS. 2 and 4 may have many similarities with cross-sections of the seat back of FIG. 1, include a center section and left and right bolster sections on transversely opposite sides of the center section. Accordingly, the configurations disclosed in FIGS. 2 and 4 are equally applicable to seat backs and seat bottoms. Further, not all seats necessarily include identifiable bolster sections, which are not required to reap the benefits of this disclosure.

In some embodiments, the externally mounted light source is directed away from the seat 10 to disinfect a neighboring interior panel, such as a door panel or console panel. In the configuration of FIG. 4, for example, the bolster-mounted light portion 22 could be made to direct light in the outboard direction and upward toward a neighboring door panel and/or door handle for disinfection while keeping the emitted light below the vehicle belt line. A forward emitting portion of the light source could be included along the front end of the seat bottom for disinfecting the brake and accelerator pedals, knee bolster area, and/or at least a portion of the steering wheel.

In another example, illustrated in FIG. 6, at least a portion of the light source 12 is located along a back side 60 of the seat back 16 and emits disinfecting light 46 toward a floor panel and/or rear door panel in a cargo area behind the seat 10, such as in an SUV or hatchback type of vehicle. This configuration is useful for disinfection of surfaces of cargo being transported by the vehicle, such as groceries or other items brought into the vehicle from a shop where the state of disinfection is uncertain. As in the above-described examples, the light source 12 may be affixed to the decorative covering 34 of the seat 10 by sewing or other suitable means. In this case, the decorative covering 34 may include carpet coordinated with the cargo floor covering. A disinfection cycle may be permitted in this case when the vehicle is occupied (e.g., being driven home) provided that the controller has received information regarding the absence of people or pets in the cargo area. Such information can be provided by sensors, as noted above, or by user input.

The portion of the light source 12 illustrated in FIG. 6 may be in addition to other portions along the exterior of the seat 10 for disinfecting the seating surface 36 or other parts of the vehicle. Vehicle seats are uniquely suited to include the disinfecting light source 12 due to their relatively central location within a vehicle and their proximity to surfaces with which vehicle occupants are most likely to come into contact. The light source 12 may include portions dedicated to disinfection of power or manual seat controls, console areas, portions of the instrument panel, etc.

A disinfection cycle, including illumination of the light source 12, preferably occurs only when there are no persons or pets in the passenger cabin. Accordingly, in some embodiments, the light source 12 is prevented from emitting light when the vehicle in which the seat 10 is installed is occupied. The controller 30 may obtain information pertinent to the presence of people in the vehicle from numerous sources, such as strain gauges or other types of sensors in or around the seat 10, an in-cabin camera or motion sensor or microphone, the presence of a keyless ignition fob in the cabin, etc. The controller 30 may be configured to initiate a disinfection cycle only when none of the available information pertinent to the presence of people in the vehicle indicates the presence of people. Moreover, after a disinfection cycle is initiated, the controller may continue to monitor these sources of information and abort the disinfection cycle if the presence of a person or other animal is detected. A 30 second duration for the cycle may be suitable for effective disinfection, although the controller may be configured to perform shorter or longer cycles. In some embodiments, disinfection cycle duration is user-selectable.

Disinfection cycles are preferably performed remotely. Accordingly, a disinfection cycle may be initiated as part of a remote vehicle starting sequence, in which the controller receives a command to check for the presence of people and to then begin the disinfection cycle when the vehicle engine is started. In another example, the controller receives information pertinent to the vehicle having been driven and then turned-off. Upon receipt of this information, the controller may begin an initiation cycle once the presence of people is no longer detected and/or a particular amount of time has passed since the presence of people is not detected. In this manner, the vehicle seat and/or its neighboring vehicle interior panels can be disinfected prior to the vehicle being driven again.

It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” “such as,” and “like,” and the verbs “comprising,” “having,” “including,” and their other verb forms, when used in conjunction with a listing of one or more components or other items, are each to be construed as open-ended, meaning that that the listing is not to be considered as excluding other, additional components or items. Other terms are to be construed using their broadest reasonable meaning unless they are used in a context that requires a different interpretation.

1. A vehicle seat comprising an external disinfecting light source.

2. The vehicle seat of claim 1, further comprising a seating surface, wherein light from the light source is directed at the seating surface.

3. The vehicle seat of claim 1, wherein light from the light source is directed away from the seat toward a vehicle interior panel when installed in a vehicle.

4. The vehicle seat of claim 1, wherein light from the light source is not directed above a belt line of a vehicle when installed in the vehicle.

5. The vehicle seat of claim 1, wherein the light source is located along a bolster section of the seat.

6. The vehicle seat of claim 5, wherein light from the light source is directed at the bolster section and toward a centerline of the seat.

7. The vehicle seat of claim 1, wherein the light source is sewn to a decorative covering of the seat.

8. The vehicle seat of claim 1, wherein the light source is a strip of disinfecting lights.

9. The vehicle seat of claim 1, wherein the light source is a flexible tube light.

10. The vehicle seat of claim 1, wherein a first portion of the light source is transversely spaced from a second portion of the light source, and the first and second portions emit light toward each other.

11. The vehicle seat of claim 1, further comprising a seat bottom and a seat back, wherein the light source comprises a plurality of portions transversely spaced along the seat bottom and the seat back.

12. The vehicle seat of claim 1, wherein light from the light source includes ultraviolet light.

13. The vehicle seat of claim 1, wherein light from the light source includes visible light.

14. The vehicle seat of claim 1, wherein the light source is prevented from emitting light when a vehicle in which the seat is installed is occupied.

15. The vehicle seat of claim 1, wherein the light source is activated remotely.
